SEQUENCE LISTING (16 sequences)

SEQ ID NO: 1 - DNA, 688 bp, Homo sapiens
  source 1..688: mol_type = other DNA
attcccttactttcttaataaactcgctttcactttatggattcaactgtaattctttcttgtgtgagatccaagaaccttcttgtagggtctggattgggacccttttctggtaacatcttcctggtgaccatgaagggacaatactgaagagacccctgaccctaaggaaatagactgcagcaccaatgggccaactttggggcgatcatcttgcccagaaacatcatgttgaaactcttggtcagaggttggatgaaagctgacagggtccatccaggagcaagtttgagccttgccagttccattttgggtgctgagtggagtggcgactatagcaaacctgtgatctctggctgctgctcagaagaaacaagagggagggatgaataatgtaaaactctggatcaatattctaattctgagcctctattggaatcagctagcaaccacatatcagcttggtttcaacagtttcccagttcatgctgctgagaagttcagagtcaaacctgaatctcacctctgcaaagagcacaggactccatggcaaacgttgtatatacgcaagtcatccctggcaccacattgatttactgcaccaggctttcttcattgtgatgatgttctctctcttttctaaaaaaaaaataaaaataaaatttaaaaaatcctaaaaaaaaaaaaa

SEQ ID NO: 2 - DNA, 142 bp, Homo sapiens
  source 1..142: mol_type = other DNA
tgatctctggctgctgctcagaagaaacaagagggagggatgaataatgtaaaactctggatcaatattctaattctgagcctctattggaatcagctagcaaccacatatcagcttggtttcaacagtttcccagttcatg

SEQ ID NO: 3 - DNA, 44 bp, Homo sapiens
  source 1..44: mol_type = other DNA
ccaagctgatatgtggttgctagctgattccaatagaggctcag

SEQ ID NO: 4 - DNA, 35 bp, Homo sapiens
  source 1..35: mol_type = other DNA
ccctcttgtttcttctgagcagcagccagagatca

SEQ ID NO: 5 - DNA, 185 bp, Homo sapiens
  source 1..185: mol_type = other DNA
ctgctgagaagttcagagtcaaacctgaatctcacctctgcaaagagcacaggactccatggcaaacgttgtatatacgcaagtcatccctggcaccacattgatttactgcaccaggctttcttcattgtgatgatgttctctctcttttctaaaaaaaaaataaaaataatttaaaaaatcct

SEQ ID NO: 6 - DNA, 45 bp, Homo sapiens
  source 1..45: mol_type = other DNA
agggatgacttgcgtatatacaacgtttgccatggagtcctgtgc

SEQ ID NO: 7 - DNA, 36 bp, Homo sapiens
  source 1..36: mol_type = other DNA
ggtgagattcaggtttgactctgaacttctcagcag

SEQ ID NO: 8 - DNA, 27 bp, Homo sapiens
  source 1..27: mol_type = other DNA
ctctattggaatcagctagcaaccaca

SEQ ID NO: 9 - DNA, 27 bp, Homo sapiens
  source 1..27: mol_type = other DNA
ctgctgagaagttcagagtcaaacctg

SEQ ID NO: 10 - DNA, 27 bp, Homo sapiens
  source 1..27: mol_type = other DNA
gaataatgtaaaactctggatcaatat

SEQ ID NO: 11 - RNA, 27 nt, synthetic construct
  misc_feature 1..27: note = siRNA
  source 1..27: mol_type = other RNA
gagataaccttagtcgatcgttggtgt

SEQ ID NO: 12 - RNA, 27 nt, synthetic construct
  misc_feature 1..27: note = siRNA
  source 1..27: mol_type = other RNA
gacgactcttcaagtctcagtttggac

SEQ ID NO: 13 - RNA, 27 nt, synthetic construct
  misc_feature 1..27: note = siRNA
  source 1..27: mol_type = other RNA
cttattacattttgagacctagttata

SEQ ID NO: 14 - RNA, 27 nt, synthetic construct
  misc_feature 1..27: note = siRNA
  source 1..27: mol_type = other RNA
ctctattggaatcagctagcaaccaca

SEQ ID NO: 15 - RNA, 27 nt, synthetic construct
  misc_feature 1..27: note = siRNA
  source 1..27: mol_type = other RNA
ctgctgagaagttcagagtcaaacctg

SEQ ID NO: 16 - RNA, 27 nt, synthetic construct
  misc_feature 1..27: note = siRNA
  source 1..27: mol_type = other RNA
gaataatgtaaaactctggatcaatat

(19) United States
(12) Patent Application Publication
(10) Pub. No.: US 2025/0092394 A1
(43) Pub. Date: Mar. 20, 2025

(54) BANCR Compositions and Methods for Treating Disease

(71) Applicant: Rosebud Biosciences, Inc., Orinda, CA (US)

(72) Inventors: Kitch WIlson Wilson, Orinda, CA (US); Evan Lyall, Castro Valley, CA (US)

(73) Assignee: Rosebud Biosciences, Inc., Orinda, CA (US)

(21) Appl. No.: 18/726,595

(22) PCT Filed: Jan. 9, 2023
(86) PCT No.: PCT/US23/60322

§ 371 Date: Jul. 3, 2024

Related U.S. Application Data
(60) Provisional application No. 63/299,550, filed on Jan. 14, 2022.

(51) Int. Cl. C12N 15/113

(52) U.S. Cl. CPC C12N 15/113; C12N 2310/14

(57) ABSTRACT

Disclosed herein are compositions and methods for treating cardiomyopathies and certain cancers using drugs that target BANCR. In an aspect siRNAs that target exons 3 and 4 of BANCR are used.

The official copy of the Sequence Listing is submitted concurrently with the specification as an xml file, made with WIPO Sequence Version 2.1.0, via EFS-Web, with a file name of “RBI.005.xml”, a creation date of Dec. 8, 2022, and a size of 18 kilobytes. The Sequence Listing filed via EFS-Web is part of the specification and is incorporated in its entirety by reference herein.

### BACKGROUND

There are approximately 7,000 known rare diseases (defined in the U.S. as fewer than 200,000 affected). This includes pediatric cardiomyopathies, lysosomal storage diseases, muscular dystrophies, cystic fibrosis, Angel-man, Rett and Prader Willi syndromes, and thousands more. Although rare individually, collectively rare diseases affect approximately 350 million people worldwide, of which 50-75% are diagnosed in childhood. Greater than 80% of rare diseases are genetic in origin and begin in utero, but unfortunately 95% still lack treatment. This is an enormous problem as 30% of children with these rare diseases will not live to see their 5^{th} birthday.

BRAF-activated non-protein coding RNA is a noncoding RNA that is encoded by the BANCR gene. Aberrant expression of long non-coding RNAs (IncRNAs) can contribute significantly to tumorigenesis and progression. BRAF activated non-coding RNA (BANCR), a 688-bp four-exon transcript, was first identified in 2012 as an oncogenic long non-coding RNA in BRAFV600E melanomas cells and was found to be associated with melanoma cell migration. Apart from melanoma, growing evidence has implicated BANCR in the development and progression of a variety of other human malignancies, including retinoblastoma, lung cancer, and gastric cancer, since its discovery. The pattern of expression of BANCR varies according to the kind of cancer, acting as either a tumour suppressor or an accelerator.

BANCR exerts its effects via modulating some tumor-related signaling pathways particularly MAPK and other regulatory mechanisms such as sponging miRNAs. BANCR has been up-regulated in endometrial, gastric, breast, melanoma, and retinoblastoma. Conversely, it has been down-regulated in some other cancers such as those originated from lung, bladder, and renal tissues. In some cancer types such as colorectal cancer, hepatocellular carcinoma and papillary thyroid carcinoma, there is no agreement about BANCR expression, necessitating the importance of additional functional studies in these tissues.

### SUMMARY

Disclosed herein are regulatory RNAs for inhibiting the expression of BANCR. The regulatory RNA can be, for example, siRNA, IncRNA, miRNA, and/or antisense RNA. The regulatory RNA can be single stranded (an antisense strand) or comprise a sense strand and an antisense strand, the antisense strand comprising a region complementary to a part of an RNA encoding BANCR. The antisense strand can comprises 15 or more contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and/or SEQ ID NO: 10. The antisense strand can comprise 15, 16, 17, 18, 19, 20, 21, 22, 23, 24, 25, 26, 27, 28, 29, 30, or 31 contiguous nucleotides complementary to SEQ ID NO: 1, SEQ ID NO: 2, SEQ ID NO: 5, SEQ ID NO: 6, SEQ ID NO: 7, SEQ ID NO: 8, SEQ ID NO: 9 and/or SEQ ID NO: 10, or contiguous nucleotides from SEQ ID NO: 3, SEQ ID NO: 4, SEQ ID NO: 6 or SEQ ID NO: 7. The regulatory RNAs complementary to SEQ ID NOs: 1-10 can include Uracil nucleotides in place of Thymidine nucleotides.

Disclosed here are methods using the regulatory RNAs to inhibit BANCR in cardiomyocytes, smooth muscle cells, cardiac fibroblasts, endothelial cells, and/or pericytes. The methods disclosed herein also include methods for inhibiting the migration of cardiomyocytes, smooth muscle cells, cardiac fibroblasts, endothelial cells, and/or pericytes using regulatory RNAs (e.g., siRNA) that target BANCR. These regulatory RNAs can be single stranded (an antisense strand) or comprise a sense strand and an antisense strand, the antisense strand comprising a region complementary to a part of an RNA encoding BANCR. The antisense strand can comprises 15 or more contiguous nucleotides complementary to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and/or SEQ ID NO: 10. The antisense strand can comprise 15, 16, 17, 18, 19, 20, 21, 22, 23, 24, 25, 26, 27, 28, 29, 30, or 31 contiguous nucleotides complementary to SEQ ID NO: 1, SEQ ID NO: 2, SEQ ID NO: 5, SEQ ID NO: 6, SEQ ID NO: 7, SEQ ID NO: 8, SEQ ID NO: 9 and/or SEQ ID NO: 10, or contiguous nucleotides from SEQ ID NO: 3, SEQ ID NO: 4, SEQ ID NO: 6 or SEQ ID NO: 7.

The regulatory RNAs for BANCR can be used to treat cardiomyopathies (e.g., dilated cardiomyopathy, hypertrophic cardiomyopathy, restrictive cardiomyopathy), Brugada syndrome and other arrhythmias, hereditary angioedema, congenital heart diseases (e.g., great vessel transposition), as well as other idiopathic, genetic/familial, primary, secondary, or syndromic cardiomyopathies as described, for example, in Lipshultz et al., Cardiomyopathy in Children: Classification and Diagnosis: A Scientific Statement from the American Heart Association, Circulation 140:e9-e68 (2019), which is hereby incorporated by reference in its entirety for all purposes.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 μg, it is intended that the concentration be understood to be at least approximately or about 10 μg.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, the terms “amplification” and “amplifying” refer to a polynucleotide amplification reaction, namely, a population of polynucleotides that are replicated from one or more starting sequences. Amplifying may refer to a variety of amplification reactions, including, but not limited to, polymerase chain reaction, linear polymerase reactions, nucleic acid sequence-based amplification, rolling circle amplification and like reactions. Typically, amplification primers are used for amplification, the result of the amplification reaction being an amplicon.

As used herein, the term “benign” means something of little or no effect. For example, genetic variants can be pathogenic or benign. A “benign variant” or “benign genetic variant” is one that has little or no effect in a disease or condition, such as eye or hair color; that is, they are considered part of the normal biology of an individual or organism and thus are often referred to as “normal variants.” Benign variants can also be considered as the opposite of “pathogenic variants,” which are causal of a disease or condition. In some embodiments of the invention, it may be desirable to identify benign variants associated with a particular phenotype that do not cause disease. Such benign variants can be identified with the present invention by use of cohorts affected and unaffected by the phenotype or trait of interest such as a desirable growth characteristic in a plant crop or a particular size or coat color of a companion animal.

As used herein, the term “coding sequence” is defined to mean a portion of a nucleic acid (e.g., a gene) that encodes an amino acid sequence of a protein.

As used herein, the terms “consensus sequence” and “canonical sequence” are defined to mean an archetypical amino acid sequence against which all variants of a particular protein or sequence of interest are compared. The terms also refer to a sequence that sets forth the nucleotides that are most often present in a DNA sequence of interest. For each position of a gene, the consensus sequence gives the amino acid that is most abundant in that position in a multiple sequence alignment (MSA).

As used herein, the terms “corresponding to”, “reference to” or “relative to” are used interchangeably when used in the context of the numbering of a given amino acid or polynucleotide sequence and are defined in this context to mean the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence.

The term “detectable phenotype” includes any cellular phenotype that can be detected and used to separate or split one population or pool of cells from another. In particular embodiments, cells of interest can be selected based upon the presence of a detectable phenotype. Examples of detectable phenotypes include, but are not limited to, cell growth, cell survival, reporter gene expression, physical characteristics of the cell (e.g., shape, size, mass, and/or density), cell mobility or migration behavior, cellular appearance or morphology, and combinations thereof. In certain embodiments, a detectable phenotype is used to determine whether a genetic element is phenotypically responsive to a modulating nucleic acid element. In other embodiments, a detectable phenotype is a phenotype that is observed with one (single-mutant phenotype), two (double-mutant phenotype), three, four, five, six, seven, eight, nine, ten, or more mutations and used to identify one or a plurality of genetic elements, one or a plurality of nucleic acid elements that modulate genetic elements, and/or genetic interactions between genetic elements.

As used herein, an “effective amount” or “therapeutically effective amount” are used interchangeably, and defined to be an amount of a compound, formulation, material, or composition, as described herein effective to achieve a particular biological result.

As used herein, the term “expression level” of a gene refers to the amount of RNA transcript that is transcribed by a gene and/or the amount of protein that may be translated from an RNA transcript, e.g. mRNA. For example, for genes which encode a miRNA, the expression level may be determined through quantifying the amount of RNA transcript which is expressed, e.g. using standard methods such as quantitative PCR of a mature miRNA, microarray, or Northern blot. Alternatively, the expression level may also be determined through measuring the effect of a miRNA on a target mRNA.

As defined herein, the term “heterologous” polynucleotide or polypeptide is defined to mean any polynucleotide or polypeptide that is not naturally found in a host cell. As such, the term includes polynucleotides that are removed from a host cell, subjected to laboratory manipulation, and then reintroduced into a host cell. In some embodiments, the introduced polynucleotide expresses the heterologous polypeptide.

As used herein, the term “molecular pathway”, also called a biological pathway, is a series of interactions among molecules in a cell that leads to a certain product or a change in a cell. Such a molecular pathway can trigger the assembly of new molecules, such as a fat or protein. Molecular pathways can also turn genes on and off, or spur a cell to move. Importantly, DNA mutations in regulatory regions of the genome can cause changes in molecular pathway activity by inhibiting or activating the expression of key molecules.

As used herein, the term “expression of a gene” or “gene expression” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi).

As used herein, an “expression vector” and an “expression construct” are used interchangeably, and are both defined to be a plasmid, virus, or other nucleic acid designed for protein expression in a cell. The vector or construct is used to introduce a gene into a host cell whereby the vector will interact with polymerases in the cell to express the protein encoded in the vector/construct. The expression vector and/or expression construct may exist in the cell extrachromosomally or integrated into the chromosome. When integrated into the chromosome the nucleic acids comprising the expression vector or expression construct will be an expression vector or expression construct.

As used herein, the term “gene” refers to a DNA sequence comprising a region (transcribed region), which is transcribed into an RNA molecule (e.g. an mRNA) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites.

As used herein, “heterologous” is defined to mean the nucleic acid and/or polypeptide is not homologous to the host cell. Alternatively, “heterologous” means that portions of a nucleic acid or polypeptide that are joined together to make a combination where the portions are from different species, and the combination is not found in nature.

As used herein, the term “next generation sequencing” and/or “high throughput sequencing” and/or “deep sequencing” refer to sequencing technologies having increased throughput as compared to the traditional Sanger- and capillary electrophoresis-based approaches, for example with the ability to generate hundreds of thousands or millions of relatively short sequence reads at a time. Examples of next generation sequencing techniques include, but are not limited to, sequencing by synthesis, sequencing by ligation, and sequencing by hybridization. Examples of next generations sequencing methods include, but are not limited to, pyrosequencing as used by the GS Junior and GS FLX Systems (454 Life Sciences, Bradford, Conn.); sequencing by synthesis as used by Miseq and Solexa system (Illumina, Inc., San Diego, Calif.); the SOLiD™ (Sequencing by Oligonucleotide Ligation and Detection) system and Ion Torrent Sequencing systems such as the Personal Genome Machine or the Proton Sequencer (Thermo Fisher Scientific, Waltham, Mass.), Single Molecule, Real-Time (SMRT) Sequencing (Pacific Biosciences, Menlo Park, Calif.); and nanopore sequencing systems (Oxford Nanopore Technologies, Oxford, united Kingdom).

As used herein, is “normal” refers to a standard or usual state. As applied in biology and medicine, a “normal state” or “normal person” is what is usual or most commonly observed. For example, individuals with disease are not typically considered normal. Example usage of the term includes, but is not limited to, “normal subject,” “normal individual,” “normal organism,” “normal cohort,” “normal group,” and “normal population.” In some cases, the term “apparently healthy” is used to describe a “normal” individual. Thus, an individual that is normal as a child may not be normal as an adult if they later develop, for example, cancer, Alzheimer's disease or are exposed to health-impairing environmental factors such as toxins or radiation. Conversely, a child treated and cured of leukemia can grow up to be an apparently healthy adult. Normal can also be described more broadly as the state not under study. For example, and as used herein, a normal cohort, used in conjunction with a particular disease cohort under investigation, includes individuals without the disease being studied but can also include individuals that have another unrelated disease or condition. Further, a normal group, normal cohort, or normal population can consist of individuals of the same ethnicity or multiple ethnicities, or likewise, same age or multiple ages, all male, all female, male and female, or any number of demographic variables. As used herein, the term “normal” can mean “normal subjects” or “normal individuals.”

As used herein, the term “normal variation” refers to the spectrum of copy number variation, or frequencies of copy number variants, found in a normal cohort or normal population (see “Normal” definition). Normal variation can also refer to the spectrum of variation, or frequencies of variants, found in a normal cohort or normal population for any class of variant found in genomes, such as, but not limited to, single nucleotide variants, insertions, deletions, and inversions.

As used herein, the term “pathogenic” is generally defined as able to cause or produce disease. For example, genetic variants can be pathogenic or benign. In some cases, the term “pathogenic variant” or “pathogenic genetic variant” is more broadly used for a variant associated with or causative of a condition, which may or may or may not be a disease. In some cases, a pathogenic variant can be considered a causative variant or causative mutation, in which case the variant is causal of the disease or condition. Pathogenic variants can also be considered as the opposite of “benign variants,” which are not causal of a disease or condition.

As used herein, the terms “percentage of sequence identity” and “percentage homology” are used interchangeably and are defined to mean comparisons among polynucleotides or polypeptides, and are determined by comparing two optimally aligned sequences over a comparison window, where the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, Adv Appl Math. 2:482, 1981; by the homology alignment algorithm of Needleman and Wunsch, J Mol Biol. 48:443, 1970; by the search for similarity method of Pearson and Lipman, Proc Natl Acad Sci. USA 85:2444, 1988; by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Greene Publishing Associates, Inc. and John Wiley & Sons, Inc., (1995 Supplement). Examples of algorithms that are suitable for determining percent sequence identity and sequence similarity are the BLAST and BLAST 2.0 algorithms, which are described in Altschul et al., J. Mol. Biol. 215:403-410, 1990; and Altschul et al., Nucleic Acids Res. 25(17):3389-3402, 1977; respectively. Software for performing BLAST analyses is publicly available through the National Center for Biotechnology Information website. BLAST for nucleotide sequences can use the BLASTN program with default parameters, e.g., a wordlength (W) of 11, an expectation (E) of 10, M=5, N=−4, and a comparison of both strands. BLAST for amino acid sequences can use the BLASTP program with default parameters, e.g., a wordlength (W) of 3, an expectation (E) of 10, and the BLOSUM62 scoring matrix (see Henikoff and Henikoff, Proc Natl Acad Sci. USA 89:10915, 1989). Exemplary determination of sequence alignment and % sequence identity can also employ the BESTFIT or GAP programs in the GCG Wisconsin Software package (Accelrys, Madison WI), using default parameters provided.

As used herein, the terms “polynucleotide” or “nucleic acid’ are used interchangeably and are defined to mean two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. Preferably, such modified or synthetic nucleobases will be encoding nucleobases.

As used herein, the terms “protein”, “polypeptide,” and “peptide” are used interchangeably and are defined to mean a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids. In some embodiments of the descriptions of polypeptides, the standard single or three letter abbreviations are used for the genetically encoded amino acids (see, e.g., IUPAC-IUB Joint Commission on Biochemical Nomenclature, “Nomenclature and Symbolism for Amino Acids and Peptides,” Eur. J. Biochem. 138:9-37, 1984).

As used herein, the terms “recombinant” or “engineered” or “non-naturally occurring” are used interchangeably and are defined to mean modified polypeptides or nucleic acids which polypeptides or nucleic acids are modified in a manner that would not otherwise exist in nature, or is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

As used herein, the term “reference sequence” is defined to mean a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes to the primary sequence.

As used herein, the term “reporter” or “reporter molecule” refers to a moiety capable of being detected indirectly or directly. Reporters include, without limitation, a chromophore, a fluorophore, a fluorescent protein, a receptor, a hapten, an enzyme, and a radioisotope.

As used herein, the term “reporter gene” refers to a polynucleotide that encodes a reporter molecule that can be detected, either directly or indirectly. Exemplary reporter genes encode, among others, enzymes, fluorescent proteins, bioluminescent proteins, receptors, antigenic epitopes, and transporters.

As used herein, the term “reporter probe” refers to a molecule that contains a detectable label and is used to detect the presence (e.g., expression) of a reporter molecule. The detectable label on the reporter probe can be any detectable moiety, including, without limitation, an isotope, chromophore, and fluorophore. The reporter probe can be any detectable molecule or composition that binds to or is acted upon by the reporter to permit detection of the reporter molecule.

As used herein, the term “stem cell” is defined as a cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system), but not into extra-embryonic tissues like the placenta. A variety of stem cell types are known in the art and can be used, including for example, embryonic stem cells, adult stem cells, inducible pluripotent stem cells, hematopoietic stem cells, neural stem cells, epidermal neural crest stem cells, mammary stem cells, intestinal stem cells, mesenchymal stem cells, olfactory adult stem cells, testicular cells, and progenitor cells (e.g., neural, angioblast, osteoblast, chondroblast, pancreatic, epidermal, etc.)

As used herein, the term “stringent hybridization conditions” is defined to mean hybridizing in 50% formamide at 5×SSC at a temperature of 42° C. and washing the filters in 0.2×SSC at 60° C. (1×SSC is 0.15M NaCl, 0.015M sodium citrate.) Stringent hybridization conditions also encompasses low ionic strength and high temperature for washing, for example 0.015 M sodium chloride/0.0015 M sodium citrate/0.1% sodium dodecyl sulfate at 50° C.; hybridization with a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

As used herein, the term “substantial identity” refers to a polynucleotide or polypeptide sequence that has at least 80 percent sequence identity, at least 85 percent identity and 89 to 95 percent sequence identity, more usually at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using standard parameters, i.e., default parameters, share at least 80 percent sequence identity, preferably at least 89 percent sequence identity, at least 95 percent sequence identity or more (e.g., 99 percent sequence identity). Preferably, residue positions which are not identical differ by conservative amino acid substitutions.

As used herein, the term “trait”, in the context of biology, refers to a trait that relates to any phenotypical distinctive character of an individual member of an organism, or of an individual cell, in comparison to (any) other individual member of the same organism, or of (any) other individual cell. For example, in the current invention traits (preferably of the same character) of cells (from the same organism) are compared. Within the context of the current invention the trait can be inherited, i.e. be passed along to next generations of the organism by means of the genetic information in the organism. As used herein, the terms “trait of the same character” and “trait of said character” refer to anyone of a group of at least two traits that exist (or became apparent) for a character. For example, in case of the character “color of the flower”, phenotypical manifestations (traits) might comprise blue, red, white, and so on. In the above example blue, red and white are all different traits of the same character.

As used herein, “transfected” or “transformed” or “transduced” are defined to be a process by which exogenous nucleic acid is transferred or introduced into a host cell. A “transfected” or “transformed” or “transduced” cell is one which has been transfected, transformed or transduced with exogenous nucleic acid. The cell includes the primary subject cell and its progeny.

As used herein, the terms “wild-type” is defined to mean the form found predominantly in nature. For example, a wild-type polypeptide or polynucleotide sequence is a sequence predominantly present in an organism that can be isolated from a source in nature and which has not been intentionally modified by human manipulation.

### Regulatory RNA

RNA interference (hereinafter “RNAi”) is a method of post-transcriptional gene regulation that is conserved throughout many eukaryotic organisms. RNAi is induced by short (i.e., <30 nucleotide) double stranded RNA (“dsRNA”) molecules which are present in the cell. These short dsRNA molecules, called “short interfering RNA” or “siRNA,” cause the destruction of target RNAs which share sequence homology with the siRNA. It is believed that the siRNA and the targeted RNA bind to an “RNA-induced silencing complex” or “RISC,” which cleaves the targeted RNA. The siRNA can be recycled much like a multiple-turnover enzyme, with a single siRNA molecule capable of inducing cleavage of approximately 1000 target RNA molecules.

In an aspect, the disclosure relates to regulatory RNAs for inhibiting the expression of BANCR (BRAF-Activated Non-Protein Coding RNA). BANCR is a 4-exon transcript of 688-bp, and was first discovered as an oncogenic long non-coding RNA in BRAF^{V600E} melanomas cells related to melanoma cell migration. BANCR also promotes cardiomyocyte migration in humans and non-human primates. The sequence for human BANCR is:

(SEQ ID NO: 1)
ATTCCCTTACTTTCTTAATAAACTCGCTTTCACTTTATGGATTCA
ACTGTAATTCTTTCTTGTGTGAGATCCAAGAACCTTCTTGTAGGG
TCTGGATTGGGACCCTTTTCTGGTAACATCTTCCTGGTGACCATG
AAGGGACAATACTGAAGAGACCCCTGACCCTAAGGAAATAGACTG
CAGCACCAATGGGCCAACTTTGGGGCGATCATCTTGCCCAGAAAC
ATCATGTTGAAACTCTTGGTCAGAGGTTGGATGAAAGCTGACAGG
GTCCATCCAGGAGCAAGTTTGAGCCTTGCCAGTTCCATTTTGGGT
GCTGAGTGGAGTGGCGACTATAGCAAACCTGTGATCTCTGGCTGC
TGCTCAGAAGAAACAAGAGGGAGGGATGAATAATGTAAAACTCTG
GATCAATATTCTAATTCTGAGCCTCTATTGGAATCAGCTAGCAAC
CACATATCAGCTTGGTTTCAACAGTTTCCCAGTTCATGCTGCTGA
GAAGTTCAGAGTCAAACCTGAATCTCACCTCTGCAAAGAGCACAG
GACTCCATGGCAAACGTTGTATATACGCAAGTCATCCCTGGCACC
ACATTGATTTACTGCACCAGGCTTTCTTCATTGTGATGATGTTCT
CTCTCTTTTCTAAAAAAAAAATAAAAATAAAATTTAAAAAATCCT
AAAAAAAAAAAAA

Regulatory RNAs (e.g., siRNAs) described herein can target BANCR RNA to reduce the half-life and/or function of the BANCR IncRNA. Regulatory RNAs (e.g., siRNAs) can target exons 3 and 4 of the BANCR RNA. The exon 3 sequence of BANCR is:

(SEQ ID NO: 2)
TGATCTCTGGCTGCTGCTCAGAAGAAACAAGAGGGAGGGATGAAT
AATGTAAAACTCTGGATCAATATTCTAATTCTGAGCCTCTATTGG
AATCAGCTAGCAACCACATATCAGCTTGGTTTCAACAGTTTCCCA
GTTCATG

The regulatory RNA can be complementary to a sequence in exon 3, and can be complementary to about 15 nucleotides to about 30 contiguous nucleotides in the target. The regulatory RNA can have 90%, 95%, or 97% sequence identity with the complement to the target sequence. The regulatory RNA can also be one that hybridizes to the target sequence under stringent hybridzation conditions. Exemplary regulatory RNAs include, for example a regulatory RNA that has 15, 16, 17, 18, 19, 20, 21, 22, 23, 24, 25, 26, 27, 28, 29, or 30 contiguous nucleotides of the 44 nucleotides of SEQ ID NO: 3 or the 35 nucleotides of SEQ ID NO: 4. Exemplary regulatory RNAs include, for example a regulatory RNA that has any 21 contiguous nucleotides of the 44 nucleotides of SEQ ID NO: 3 or the 35 nucleotides of SEQ ID NO: 4.

(SEQ ID NO: 3)
CCAAGCTGAT ATGTGGTTGC TAGCTGATTC CAATAGAGGC TCAG
(SEQ ID NO: 4)
CCCTCTTGTT TCTTCTGAGC AGCAGCCAGA GATCA

The exon 4 sequence of BANCR is:

(SEQ ID NO: 5)
CTGCTGAGAAGTTCAGAGTCAAACCTGAATCTCACCTCTGCAAAG
AGCACAGGACTCCATGGCAAACGTTGTATATACGCAAGTCATCCC
TGGCACCACATTGATTTACTGCACCAGGCTTTCTTCATTGTGATG
ATGTTCTCTCTCTTTTCTAAAAAAAAAATAAAAATAATTTAAAAA
ATCCT

The regulatory RNA can be complementary to a sequence in exon 4, and can be complementary to about 15 nucleotides to about 30 contiguous nucleotides in the target. The regulatory RNA can have 90%, 95%, or 97% sequence identity with the complement to the target sequence. The regulatory RNA can also be one that hybridizes to the target sequence under stringent hybridzation conditions. Exemplary regulatory RNAs include, for example a regulatory RNA that has 15, 16, 17, 18, 19, 20, 21, 22, 23, 24, 25, 26, 27, 28, 29, or 30 contiguous nucleotides of the 45 nucleotides of SEQ ID NO: 6 or the 36 nucleotides of SEQ ID NO: 7. Exemplary regulatory RNAs include, for example a regulatory RNA that has any 21 contiguous nucleotides of the 45 nucleotides of SEQ ID NO: 6 or the 36 nucleotides of SEQ ID NO: 7.

(SEQ ID NO: 6)
AGGGATGACT TGCGTATATA CAACGTTTGC CATGGAGTCC TGTGC
(SEQ ID NO: 7)
GGTGAGATTC AGGTTTGACT CTGAACTTCT CAGCAG

In an aspect, the disclosure describes isolated siRNA comprising short double-stranded RNA from about 15 nucleotides to about 30 nucleotides in length, or between 18 and 25, or 18, 19, 20, 21, 22, 23, 24, or 25 nucleotides in length and are targeted to the target mRNA. The siRNA comprise a sense RNA strand and a complementary antisense RNA strand annealed together by standard Watson-Crick base-pairing interactions (hereinafter “base-paired”). Each strand of the duplex can be the same length or of different lengths. As is described in more detail below, the sense strand comprises a nucleic acid sequence which is identical to a target sequence contained within the target mRNA.

The sense and antisense strands of a siRNA can comprise two complementary, single-stranded RNA molecules or can comprise a single molecule in which two complementary portions are base-paired and are covalently linked, for example, by a single-stranded hairpin loop. Without wishing to be bound by any theory, it is believed that the hairpin loop of the latter type of siRNA molecule is cleaved intracellularly by the Dicer protein (or its equivalent) to form an siRNA of two individual base-paired RNA molecules.

siRNA can comprise partially purified RNA, substantially pure RNA, synthetic RNA, recombinantly produced RNA, as well as altered RNA that differs from naturally-occurring RNA by the addition, deletion, substitution and/or alteration of one or more nucleotides, or combinations of one or more of the foregoing. Alterations can include addition of non-nucleotide material, such as to the end(s) of the siRNA or to one or more internal nucleotides of the siRNA, including modifications that make the siRNA resistant to nuclease digestion. One or both strands of the siRNA can also comprise a 3′ overhang. As used herein, a 3′ overhang refers to at least one unpaired nucleotide extending from the 3′-end of a duplexed RNA strand. The 3′ overhang can have 1 to about 6 nucleotides (which includes ribonucleotides or deoxynucleotides) in length, or from 1 to about 5 nucleotides in length, or from 1 to about 4 nucleotides in length, or from about 2 to about 4 nucleotides in length. The 3′ overhang can be present on both strands of the siRNA, and can be 2 nucleotides in length. For example, each strand of an siRNA can have 3′ overhangs of dithymidylic acid (TT) or diuridylic acid (UU).

In order to enhance the stability of a siRNA, the 3′ overhangs can be stabilized against degradation. For example, the overhangs can be stabilized by including purine nucleotides, such as adenosine or guanosine nucleotides. The overhangs can also be stabilized by substitution of pyrimidine nucleotides with modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

The siRNA can have the sequence AA(N19)TT or NA(N21), where N is any nucleotide. These siRNA can have approximately 30-70% G/C content, and can comprise approximately 50% G/C content. The sequence of the sense siRNA strand can correspond to (N19)TT or N21 (i.e., positions 3 to 23), respectively. In the latter case, the 3′ end of the sense siRNA can be converted to TT. The rationale for this sequence conversion is to generate a symmetric duplex with respect to the sequence composition of the sense and antisense strand 3′ overhangs. The antisense RNA strand can then synthesized as the complement to positions 1 to 21 of the sense strand.

When Position 1 of the 23-nt sense strand is not recognized in a sequence-specific manner by the antisense strand, the 3′-most nucleotide residue of the antisense strand can be chosen deliberately. However, in this case the penultimate nucleotide of the antisense strand (complementary to position 2 of the 23-nt sense strand in either embodiment) is generally complementary to the targeted sequence.

The siRNA can also have the sequence NAR(N17)YNN, where R is a purine (e.g., A or G) and Y is a pyrimidine (e.g., C or U/T). The respective 21-nt sense and antisense RNA strands therefore generally begin with a purine nucleotide. Such siRNA can be expressed from pol III expression vectors without a change in targeting site, as expression of RNAs from pol III promoters is only believed to be efficient when the first transcribed nucleotide is a purine.

The siRNA usually has a sequence having no more than five (5) consecutive purines or pyrimidines. The siRNA also usually comprises a sequence having no more than five (5) consecutive nucleotides having the same nucleobase (i.e., A, C, G, or U/T).

The siRNA can be targeted to any stretch of approximately 19-25 contiguous nucleotides in any of the target mRNA sequences (the “target sequence”). Techniques for selecting target sequences for siRNA are given, for example, in Fakhr et al., Precise and efficient siRNA design: a key point in competent gene silencing, Cancer Gene Therapy 23:73-82 (2016), which is hereby incorporated by reference in its entirety for all purposes. Thus, the sense strand of the present siRNA comprises a nucleotide sequence identical to any contiguous stretch of about 19 to about 25 nucleotides in the target mRNA.

The siRNA can be obtained using a number of techniques known to those of skill in the art. For example, the siRNA can be chemically synthesized or recombinantly produced using methods known in the art, such as the Drosophila in vitro system described in U.S. published application 2002/0086356, which is hereby incorporated by reference in its entirety for all purposes. siRNA can be chemically synthesized using appropriately protected ribonucleoside phosphoramidites and a conventional DNA RNA synthesizer. The siRNA can be synthesized as two separate, complementary RNA molecules, or as a single RNA molecule with two complementary regions. Commercial suppliers of synthetic RNA molecules or synthesis reagents are well known in the art.

siRNA can also be expressed from recombinant circular or linear DNA plasmids using any suitable promoter. Suitable promoters for expressing siRNA from a plasmid include, for example, the U6 or H1 RNA pol III promoter sequences and the cytomegalovirus promoter. Selection of other suitable promoters is within the skill in the art. Recombinant plasmids can also comprise inducible or regulatable promoters for expression of the siRNA in a particular tissue or in a particular intracellular environment.

The siRNA expressed from recombinant plasmids can either be isolated from cultured cell expression systems by standard techniques, or can be expressed intracellularly at or near a target tissue or cells in vivo. siRNA can be expressed from a recombinant plasmid either as two separate, complementary RNA molecules, or as a single RNA molecule with two complementary regions. Selection of plasmids suitable for expressing siRNA, methods for inserting nucleic acid sequences for expressing the siRNA into the plasmid, and methods of delivering the recombinant plasmid to the cells of interest are within the skill in the art. See, for example Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16: 948-958; Lee N S et al. (2002), Nat. Biotechnol. 20: 500-505; and Paul C P et al. (2002), Nat. Biotechnol. 20: 505-508, all of which are incorporated by reference in their entirety for all purposes.

siRNA can also be expressed from recombinant viral vectors intracellularly at or near the target tissue or cells in vivo. The recombinant viral vectors can comprise sequences encoding the siRNA and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors can also comprise inducible or regulatable promoters for expression of the siRNA in a particular tissue or in a particular intracellular environment. siRNA can be expressed from a recombinant viral vector either as two separate, complementary RNA molecules, or as a single RNA molecule with two complementary regions.

Any viral vector capable of accepting the coding sequences for the siRNA molecule(s) to be expressed can be used for example vectors derived from adenovirus (AV); adeno-associated virus (AAV); retroviruses (e.g., lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

The siRNA can be chemically modified to enhance stability. The siRNA may be synthesized and/or modified by methods well established in the art, such as those described in “Current protocols in nucleic acid chemistry,” Beaucage, S. L. et al. (Eds.), John Wiley & Sons, Inc., New York, N.Y., USA, which is hereby incorporated herein by reference. Specific examples of siRNA compounds include siRNAs containing modified backbones or no natural intemucleoside linkages. siRNAs having modified backbones include those that retain a phosphorus atom in the backbone and those that do not have a phosphorus atom in the backbone. Modified siRNAs that do not have a phosphorus atom in their internucleoside backbone can also be considered to be oligonucleosides.

Modified siRNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; and 5,625,050, each of which is incorporated by reference in itsd entirety for all purposes.

Modified siRNA backbones that do not include a phosphorus atom therein have backbones that are formed by short chain alkyl or cycloalkyl intemucleoside linkages, mixed heteroatoms and alkyl or cycloalkyl intemucleoside linkages, or one or more short chain heteroatomic or heterocyclic intemucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other suitable siRNA mimetics, both the sugar and the internucleoside linkage, i.e., the backbone, of the nucleotide units are replaced with novel groups. The base units are maintained for hybridization with an appropriate nucleic acid target compound. One such oligomeric compound, a dsRNA mimetic that has been shown to have excellent hybridization properties, is referred to as a peptide nucleic acid (PNA). In PNA compounds, the sugar backbone of a siRNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500, which is incorporated by reference in its entirety for all purposes.

In another aspect, siRNAs can have phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. Also preferred are dsRNAs having morpholino backbone structures of the above-referenced U.S. Pat. No. 5,034,506.

Modified siRNAs may also contain one or more substituted sugar moieties. siRNAs can comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH.sub.2, and O(CH₂)ₙON[(CH₂)ₙCH₃)]₂, where n and m are from 1 to about 10. Other preferred dsRNAs comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an dsRNA, or a group for improving the pharmacodynamic properties of an dsRNA, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxy-alkoxy group. A further preferred modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications can include 2′-methoxy (2′-OCH₃), 2′-aminopropoxy (2′-OCH₂CH₂CH₂NH₂) and 2′-fluoro (2′-F). Similar modifications may also be made at other positions on the siRNA, particularly the 3′ position of the sugar on the 3′ terminal nucleotide or in 2′-5′ linked siRNAs and the 5′ position of 5′ terminal nucleotide. siRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is incorporated by reference in its entirety for all purposes.

siRNAs may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, DsRNA Research and Applications, pages 289-302, Crooke, S. T. and Lebleu, B., Ed., CRC Press, 1993, each of which is incorporated by reference in its entirety for all purposes. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., DsRNA Research and Applications, CRC Press, Boca Raton, 1993, pp. 276-278, which is incorporated by reference in its entirety for all purposes) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941, and U.S. Pat. No. 5,750,692, each of which is incorporated by reference in its entirety for all purposes.

Another modification of the siRNAs can involve chemically linking to the siRNA one or more moieties or conjugates which enhance the activity, cellular distribution or cellular uptake of the siRNA. Such moieties include but are not limited to lipid moieties such as a cholesterol moiety (Letsinger et al., Proc. Natl. Acid. Sci. USA, 1989, 86:6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937), each of the foregoing references are incorporated by reference in its entirety for all purposes.

Representative U.S. patents that teach the preparation of such siRNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference in its entirety for all purposes.

It is not necessary for all positions in a given compound to be uniformly modified, and in fact more than one of the aforementioned modifications may be incorporated in a single compound or even at a single nucleoside within a siRNA. The present invention also includes dsRNA compounds which are chimeric compounds. “Chimeric” siRNA compounds or “chimeras,” in the context of this invention, are siRNA compounds, particularly siRNAs, which contain two or more chemically distinct regions, each made up of at least one monomer unit, i.e., a nucleotide in the case of a siRNA compound. These siRNAs typically contain at least one region wherein the siRNA is modified so as to confer upon the siRNA increased resistance to nuclease degradation, increased cellular uptake, and/or increased binding affinity for the target nucleic acid. An additional region of the siRNA may serve as a substrate for enzymes capable of cleaving RNA:DNA or RNA:RNA hybrids. By way of example, RNase His a cellular endonuclease which cleaves the RNA strand of an RNA:DNA duplex. Activation of RNase H, therefore, results in cleavage of the RNA target, thereby greatly enhancing the efficiency of siRNA inhibition of gene expression. Consequently, comparable results can often be obtained with shorter siRNAs when chimeric siRNAs are used, compared to phosphorothioate deoxysiRNAs hybridizing to the same target region.

In certain instances, the siRNA may be modified by a non-ligand group. A number of non-ligand molecules have been conjugated to siRNAs in order to enhance the activity, cellular distribution or cellular uptake of the siRNA, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923), each of he foregoing references is incorporated by reference in its entirety for all purposes. Representative United States patents that teach the preparation of such siRNA conjugates have been listed above. Typical conjugation protocols involve the synthesis of siRNAs bearing an aminolinker at one or more positions of the sequence. The amino group is then reacted with the molecule being conjugated using appropriate coupling or activating reagents. The conjugation reaction may be performed either with the siRNA still bound to the solid support or following cleavage of the siRNA in solution phase. Purification of the siRNA conjugate by HPLC typically affords the pure conjugate.

Liposomes can aid in the delivery of the siRNA to a target tissue or cell, such as cardiomyocytes, smooth muscle cells, cardiac fibroblasts, endothelial cells, and/or pericytes, and can also increase the blood half-life of the siRNA. Liposomes suitable for use can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al. (1980), Ann. Rev. Biophys. Bioeng. 9: 467′ and U.S. Pat. Nos. 4,235,871, 4,501,728, 4,837,028, and 5,019,369, all of which are incorporated by reference in their entirety for all purposes. The liposomes encapsulating the siRNA can include a ligand molecule that can target the liposome to a targetr cell or tissue at or near the site of the tissue or cells to be treated. Ligands which bind to receptors prevalent in target cells, such as monoclonal antibodies that bind to tissue or cell specific antigens can include, for example, cardiomyocytes, smooth muscle cells, cardiac fibroblasts, endothelial cells, and/or pericytes.

The liposomes encapsulating the siRNA can be modified so as to avoid clearance by the mononuclear macrophage and reticuloendothelial systems, for example by having opsonization-inhibition moieties bound to the surface of the structure. A liposome can comprise both opsonization-inhibition moieties and a ligand. Opsonization-inhibiting moieties can be large hydrophilic polymers that are bound to the liposome membrane. As used herein, an opsonization inhibiting moiety is bound to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, which is incorporated by reference in its entirety for all purposes. Liposomes modified with opsonization-inhibition moieties thus remain in the circulation longer than unmodified liposomes.

Opsonization inhibiting moieties for modifying liposomes can include, for example, water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols. e.g., polyvinylalcohol and polyxylitol to which carboxylic or amino groups are chemically linked, as well as gangliosides, such as ganglioside GM₁. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

The opsonization-inhibiting moiety can be a PEG, PPG, or derivatives thereof. Liposomes modified with PEG or PEG-derivatives can be called PEGylated liposomes. The opsonization inhibiting moiety can be bound to the liposome membrane by any one of numerous well-known chemistries. For example, the chemistries described in Banerjee et al., Poly(ethylene glycol)-prodrug conjugates: concept, design and applications, J. Drug Delivery 2012:103973, doi: 10.1155/2012/103973, which is incorporated by reference in its entirety for all purposes.

### Cellular Differentiation

hiPSCs (100) can be differentiated into the cell type or tissue of interest using established protocols (see Indications section below for a comprehensive list of cell types with published protocols). Examples of differentiation protocols include:

Cardiomyocytes. An example protocol for cardiomyocyte differentiation (Burridge, P.W., Matsa, E., Shukla, P., et al. (2014). Chemically defined generation of human cardiomyocytes. Nat Methods 11, 855-860, which is incorporated by reference in its entirety for all purposes). Chemically defined generation of human cardiomyocytes. Nat Methods 11, 855-860, which is incorporated by reference in its entirety for all purposes) is as follows. Briefly, differentiation medium consisting of RPMI-1640 media (Life Technologies) supplemented with B27® minus insulin (Life Technologies) (RPMI+B27 minus) is used. To this medium, various small molecules are added over a week-long timetable as previously described (Burridge, P.W., Matsa, E., Shukla, P., et al. (2014). Chemically defined generation of human cardiomyocytes. Nat Methods 11, 855-860, which is incorporated by reference in its entirety for all purposes). On the first day (D0) of hiPSC differentiation, 6 μM CHIR 99021 (LC Laboratories) is added. On D2, the medium is aspirated and replaced with RPMI+B27 minus. On D3, the medium is aspirated and replaced with 5 μM of IWR-1 (Selleck Chemicals) in RPMI+B27 minus. The medium is replaced with RPMI+B27 minus on D5 and RPMI plus B27 supplemented with insulin (Life Technologies) (RPMI+B27) on D7. Cardiomyocytes can be maintained in RPMI+B27 with media change every other day. Cardiomyocytes generally begin spontaneously beating between D7-D10. A glucose starvation step further purifies cardiomyocyte culture if needed.

### Organoid Differentiation

Standard hiPSC differentiation methods often yield homogenous differentiated cells in monolayers or sheets without multilineage organoid or embryoid organization. Organoids are more complex than homogenous cell cultures, and can better mimic the biology of human tissues and organs (Kim, J., Koo, B.-K., and Knoblich, J.A. (2020). Human organoids: model systems for human biology and medicine. Nat Rev Mol Cell Biol 21, 571-584, which is incorporated by reference in its entirety for all purposes). Such organoid differentiation methods may include hiPSC-derived 2D or 3D fetal discoids, spheroids, organoids, and engineered artificial tissues that contain cells from multiple lineages (e.g. three embryonic germ layer formation (Warmflash, A., Sorre, B., Etoc, F., et al. (2014). A method to recapitulate early embryonic spatial patterning in human embryonic stem cells. Nat Methods 11, 847-854, which is incorporated by reference in its entirety for all purposes) and even vasculature) and are differentiated from hiPSCs using established methods (Warmflash, A., Sorre, B., Etoc, F., et al. (2014). A method to recapitulate early embryonic spatial patterning in human embryonic stem cells. Nat Methods 11, 847-854; Wilson, K.D., Ameen, M., Guo, H., et al. (2020). Endogenous retrovirus-derived IncRNA BANCR promotes cardiomyocyte migration in humans and non-human primates. Dev Cell 54, 694-709. Each is incorporated by reference in its entirety for all purposes). hiPSCs can also be differentiated into embryoids that contain all three embryonic germ layers (mesoderm, endoderm, and ectoderm) that mimic development of a human embryo in utero.

Wilson, K.D., Ameen, M., Guo, H., et al. (2020). Endogenous retrovirus-derived IncRNA BANCR promotes cardiomyocyte migration in humans and non-human primates. Dev Cell 54, 694-709. Wilson et al. use primate hESC and hiPSC-derived cardiomyocytes that mimic fetal cardiomyocytes in vitro to discover hundreds of novel mRNA transcripts from the primate-specific MER41 family, some of which are regulated by the cardiogenic transcription factor TBX5. The most significant of these are located within BANCR, a long non-coding RNA (IncRNA) exclusively expressed in primate fetal cardiomyocytes. Functional studies using geometrically-patterned hiPSC and hESC-derived cardiac organoids (“cardioids”) revealed that BANCR promotes cardiomyocyte migration in vitro and ventricular enlargement in vivo. They conclude that recently evolved TE loci such as BANCR may represent potent de novo developmental regulatory elements that can be interrogated with species-matching pluripotent stem cell models.

### Indications

Regulatory RNAs (e.g., siRNA) targeting BANCR and compositions containing at least one such regulatory RNA for the treatment of a BANCR-mediated disorder or disease. For example, a siRNA targeting a BANCR gene can be useful for the treatment of dilated cardiomyopathy.

The disclosure features a method of administering a siRNA targeting BANCR to a patient having a disease or disorder mediated by BANCR expression, such as a dilated cardiomyopathy. Administration of the siRNA can be via intravenous infusion, minimally invasive intramuscular and/or intracoronary delivery via cardiac catheterization, or direct cardiac injection via thoracotomy in a patient with dilated cardiomyopathy. Patients can be administered a therapeutic amount of siRNA, such as 0.1 mg/kg, 0.2 mg/kg, 0.5 mg/kg, 1.0 mg/kg, 1.5 mg/kg, 2.0 mg/kg, or 2.5 mg/kg dsRNA. The siRNA can be administered by intravenous infusion over a period of time, such as over a 5 minute, 10 minute, 15 minute, 20 minute, 25 minute, 60 minute, 120 minute or 180 minute period. The administration can be repeated, for example, on a regular basis, such as biweekly (i.e., every two weeks) for one month, two months, three months, four months or longer. After an initial treatment regimen, the treatments can be administered on a less frequent basis. For example, after administration biweekly for three months, administration can be repeated once per month, for six months or a year or longer. Administration of the siRNA can reduce BANCR levels in the myocardium of the patient by at least 20%, 25%, 30%, 40%, 50%, 60%, 70%, 80% or 90% or more.

Many BANCR-associated diseases and disorders are hereditary. Therefore, a patient in need of a BANCR siRNA can be identified by taking a family history. A healthcare provider, such as a doctor, nurse, or family member, can take a family history before prescribing or administering a BANCR siRNA. A DNA test may also be performed on the patient to identify a mutation in the BANCR gene, before a BANCR siRNA is administered to the patient.

Other indications that can be treated include, for example, other cardiomyopathies (e.g., Sun N, et al. Patient-specific induced pluripotent stem cells as a model for familial dilated cardiomyopathy. Science Translational Medicine. 2012;4:130ra47. Lan F, et al. Abnormal calcium handling properties underlie familial hypertrophic cardiomyopathy pathology in patient-specific induced pluripotent stem cells. Cell Stem Cell. 2013; 12:101-13, each is incorporated by reference in its entirety for all purposes), Brugada syndrome (Liang P, et al. Patient-specific and genome-edited induced pluripotent stem cell-derived cardiomyocytes elucidate single cell phenotype of Brugada Syndrome. J Am Coll Cardiol. 2016;68:2086-2096, which is incorporated by reference in its entirety for all purposes) and other arrhythmias, hereditary angioedema, Tetralogy of Fallot (Grunert M, et al. Induced pluripotent stem cells of patients with Tetralogy of Fallot reveal transcriptional alterations in cardiomyocyte differentiation. Scientific Reports. 2020;10, which is incorporated by reference in its entirety for all purposes), great vessel transposition, other congenital diseases. Differentiation of hiPSCs includes cardiomyocytes (Burridge, P.W., Matsa, E., Shukla, P., et al. (2014). Chemically defined generation of human cardiomyocytes. Nat Methods 11, 855-860, which is incorporated by reference in its entirety for all purposes). Chemically defined generation of human cardiomyocytes. Nat Methods 11, 855-860, which is incorporated by reference in its entirety for all purposes), endothelial cells, smooth muscle cells, cardiac fibroblasts, multicellular 2D beating organoids (Myers, F.B., Silver, J.S., Zhuge, Y., et al. (2013). Robust pluripotent stem cell expansion and cardiomyocyte differentiation via geometric patterning. Integr Biol 5, 1495-1506, which is incorporated by reference in its entirety for all purposes), or multicellular 3D organoids and engineered heart tissues that may include blood vessels. Disease phenotypic monitoring during and after differentiation may include live cell microscopy, immunophenotyping, flow cytometry, FACS, electrophysiologic measurements, calcium dynamics, contraction and sarcomeric measurements, migration, angiogenic vessel formation, morphology and function.

Cancers can also be treated including, for example, lymphoblastic and myeloid leukemias (Papapetrou EP. Modeling leukemia with human induced pluripotent stem cells. Cold Spring Harb Perspect Med. 2019;9:a034868, which is incorporated by reference in its entirety for all purposes), lymphomas, neuroblastoma, glioblastoma, Ewing's sarcoma, osteosarcoma (Lin Y-H, et al. Osteosarcoma: Molecular Pathogenesis and hiPSC Modeling. Trends in Molecular Medicine. 2017;23:737-755, which is incorporated by reference in its entirety for all purposes), Wilms tumor, rhabdomyosarcoma, retinoblastoma, spinal cord tumors, Li-Fraumeni syndrome (Zhou R, et al. Li-Fraumeni Syndrome disease model: A platform to develop precision cancer therapy targeting oncogenic p53. Trends in Pharmacological Sciences. 2017;38:908-927, which is incorporated by reference in its entirety for all purposes). BANCR has previously been shown to play a role in the pathogenesis of cancers such as lung cancer, gastric cancer, colorectal cancer, melanoma, thyroid cancer, osteosarcoma, retinoblastoma, and hepatocellular carcinoma (Yu X, et al. BANCR: a cancer-related long non-coding RNA. Am J Cancer Res. 2017;7(9):1779-1787, which is incorporated by reference in its entirety for all purposes). Disease models include all cell types, tissues and organs specifically affected by these cancers. As many cancers may affect multiple organs, disease models may also include embryonic differentiation (embryoids or embryoid bodies) that contain cells derived from all three embryonic germ layers. To mimic the potential for stem cell-based etiology of cancers, hiPSC-derived cells and tissues may be repogrammed back to undifferentiated hiPSCs using Yamanaka factor methods, and then re-differentiated to the cell type or tissue of interest. Multiple cycles of differentiation/repogramming may be required to elicit the cancer phenotype. Disease phenotypic monitoring during and after differentiation likewise includes cell type-specific, tissue-specific and organ-specific assays of health and disease tailored to the specific cancer. In general this includes assays for cellular invasion, migration, morphology, immunophenotyping, nuclear-to-cytoplasm ratios, cytogenetics and/or DNA mutation monitoring, mitosis and cellular division. To elicit the cancer phenotype in hiPSC disease models, external stressors such as ionizing radiation may be applied.

### Polynucleotides and Expression Vectors

In an aspect, polynucleotides can encode any of the engineered RNAs described herein. The polynucleotides may be operatively linked to one or more control sequences that control gene expression to create a recombinant polynucleotide capable of expressing the polypeptide. Expression constructs containing a heterologous polynucleotide encoding the engineered RNAs can be introduced into appropriate host cells to express the corresponding inhibitory RNA.

Accordingly, in some aspects, the polynucleotide encodes an engineered inhibitory RNA having at least 60%, 65%, 70%, 75%, 80%, 85%, 90%, 91%, 92%, 93%, 94%, 95%, 96%, 97%, 98%, 99% or more sequence identity to an RNA that is complementary to a 15, 16, 17, 18, 19, 20, 21, 22, 23, 24, 25, 26, 27, 28, 29, or 30 contiguous nucleotides if SEQ ID NOs: 1, 2, or 5, or contiguous nucleotides of SEQ ID NOs: 3, 4, 6, or 7. The polynucleotide can encode an inhibitory RNA that binds under stringent hybridization conditions to 15, 16, 17, 18, 19, 20, 21, 22, 23, 24, 25, 26, 27, 28, 29, or 30 contiguous nucleotides of SEQ ID NOs: 1, 2, or 5.

The polynucleotides can be capable of hybridizing under highly stringent conditions to a reference polynucleotide sequence selected from SEQ ID NO: 1, 2, or 5, or a complement thereof, and encodes a siRNA or other RNA having inhibitory activity for BANCR, with one or more of the improved properties described herein.

In another aspect, the polynucleotide encoding an inhibitory RNA may be manipulated in a variety of ways to provide for expression of the RNA. The polynucleotides encoding the inhibitory RNA can be provided as expression vectors where one or more control sequences are present to regulate the expression of the polynucleotides. Manipulation of the isolated polynucleotide prior to its insertion into a vector may be desirable or necessary depending on the expression vector. The techniques for modifying polynucleotides utilizing recombinant DNA methods are well known in the art. Guidance is provided in Sambrook et al., Molecular Cloning: A Laboratory Manual, 3^{rd} Ed., Cold Spring Harbor Laboratory Press (2001); and Current Protocols in Molecular Biology, Ausubel. F. ed., Greene Pub. Associates, (1998), with updates to 2006.

In an aspect, the control sequences include among others, promoters, enhancers, leader sequences, polyadenylation sequences, propeptide sequences, signal peptide sequences, and transcription terminators. Other control sequences will be apparent to the person of skill in the art.

Exemplary promoters for mammalian cells include, among others, CMV IE promoter, elongation factor 1α-subunit promoter, ubiquitin C promoter, Simian Virus 40 promoter, and phosphoglycerate Kinase-1 promoter.

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound.

In another aspect, the present disclosure is also directed to a recombinant expression vector comprising a polynucleotide encoding an engineered inhibitory RNA, and one or more expression regulating regions such as a promoter and a terminator, a replication origin, etc., depending on the type of hosts into which they are to be introduced.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The expression vector can exist as a single copy in the host cell, or maintained at higher copy numbers, e.g., up to 4 for low copy number and 50 or more for high copy number.

In some aspects, the expression vector contains one or more selectable markers, which permit selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol (Example 1) or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

### Host Cells

In another aspect, the present disclosure provides a host cell comprising a polynucleotide encoding an engineered inhibitory RNA of the present disclosure. Host cells can be prokaryotic or eukaryotic cells. Prokaryotic host cells include eubacteria such as, for example, Bacillus, such as B. lichenformis or B. subtilis; Pantoea, such as P. citrea; Pseudomonas, such as P. alcaligenes; Streptomyces, such as S. lividans or S. rubiginosus; Escherichia, such as E. coli; Enterobacter; Streptococcus; Archaea, such as Methanosarcina mazei; or Corynebacterium, such as C. glutamicum. The host cell can be a gram-positive bacterium such as, for example, strains of Streptomyces (e.g., s. lividans, S. coelicolor, or S. griseus) and Bacillus. The host cell can also be a gram-negative bacterium, such as, for example, E. coli or Pseudomonas sp.

Eukaryotic host cells can include, for example, fungi, algal, plant, or mammalian cells. Fungal host cells include, for example, Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizobus oryzae, Yarrowia lipolytica, and the like.

Examples of algal host cells include, for example, green algae, red algae, glaucophytes, chlorarachniophytes, euglenids, chromista, dinoflagellates, Chlorella, Chlamydomonas, Scenedesmus, Isochrysis, Dunaliella, Tetraselmis, Nannochloropsis, or Prototheca.

Plant host cells include, for example, cells of monocotyledonous or dicotyledonous plants including, but not limited to, maize, wheat, barley, rye, oat, rice, soybean, peanut, pea, lentil and alfalfa, cotton, rapeseed, canola, pepper, sunflower, potato, tobacco, tomato, eggplant, eucalyptus, a tree, an ornamental plant, a perennial grass, or a forage crop. In other embodiments the host cells are algal, including but not limited to algae of the genera,

Polynucleotides for expression of the inhibitory RNA may be introduced into cells by various methods known in the art. Techniques include among others, electroporation, biolistic particle bombardment, liposome mediated transfection, calcium chloride transfection, microinjection, recombinant viral transfection, and protoplast fusion. The introduced nucleic acids may be integrated into chromosomal DNA or maintained as extrachromosomal replicating sequences. General transformation techniques are known in the art (see, e.g., Current Protocols in Molecular Biology, F. M. Ausubel et al. eds, Chapter 9 (1987); Sambrook et al., Molecular Cloning: A Laboratory Manual, 3rd Ed., Cold Spring Harbor Laboratory Press, N.Y. (2001); and Campbell et al., Curr Genet. 16:53-56, 1989; each publication incorporated herein by reference).

### Phamaceutical Compositions

Pharmaceutical compositions containing a siRNA, as described herein, and a pharmaceutically acceptable carrier. The pharmaceutical composition containing the siRNA is useful for treating a disease or disorder associated with the expression or activity of BANCR, such as pathological processes mediated associated with BANCR. Such pharmaceutical compositions are formulated based on the mode of delivery. One example is compositions that are formulated for systemic administration via parenteral delivery, e.g., by intravenous (IV) delivery. Another example is compositions that are formulated for direct delivery into the brain parenchyma, e.g., by infusion into the brain, such as by continuous pump infusion.

The pharmaceutical compositions featured herein are administered in dosages sufficient to inhibit expression of BANCR.

In general, a suitable dose of siRNA will be in the range of 0.01 to 200.0 milligrams per kilogram body weight of the recipient per day, generally in the range of 1 to 50 mg per kilogram body weight per day. For example, the dsRNA can be administered at 0.0059 mg/kg, 0.01 mg/kg, 0.0295 mg/kg, 0.05 mg/kg, 0.0590 mg/kg, 0.163 mg/kg, 0.2 mg/kg, 0.3 mg/kg, 0.4 mg/kg, 0.5 mg/kg, 0.543 mg/kg, 0.5900 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.628 mg/kg, 2 mg/kg, 3 mg/kg, 5.0 mg/kg, 10 mg/kg, 20 mg/kg, 30 mg/kg, 40 mg/kg, or 50 mg/kg per single dose.

the dosage can be between 0.01 and 0.2 mg/kg. For example, the dsRNA can be administered at a dose of 0.01 mg/kg, 0.02 mg/kg, 0.3 mg/kg, 0.04 mg/kg, 0.05 mg/kg, 0.06 mg/kg, 0.07 mg/kg 0.08 mg/kg 0.09 mg/kg, 0.10 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, or 0.20 mg/kg. The dosage can be between 0.005 mg/kg and 1.628 mg/kg. For example, the dsRNA can be administered at a dose of 0.0059 mg/kg, 0.0295 mg/kg, 0.0590 mg/kg, 0.163 mg/kg, 0.543 mg/kg, 0.5900 mg/kg, or 1.628 mg/kg. The dosage can be between 0.2 mg/kg and 1.5 mg/kg. For example, the dsRNA can be administered at a dose of 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, or 1.5 mg/kg.

The pharmaceutical composition may be administered once daily, or the siRNA may be administered as two, three, or more sub-doses at appropriate intervals throughout the day or even using continuous infusion or delivery through a controlled release formulation. In that case, the siRNA contained in each sub-dose must be correspondingly smaller in order to achieve the total daily dosage. The dosage unit can also be compounded for delivery over several days, e.g., using a conventional sustained release formulation which provides sustained release of the siRNA over a several day period. Sustained release formulations are well known in the art and are particularly useful for delivery of agents at a particular site, such as could be used with the agents of the present invention. In this embodiment, the dosage unit contains a corresponding multiple of the daily dose.

Treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model, as described elsewhere herein.

The pharmaceutical compositions can be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; or intracranial, e.g., intraparenchymal, intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Suitable topical formulations include those in which the dsRNAs featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearoylphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). DsRNAs featured in the invention may be encapsulated within liposomes or may form complexes thereto, in particular to cationic liposomes. Alternatively, dsRNAs may be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C.sub.1-10 alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

There are many organized surfactant structures besides microemulsions that have been used for the formulation of regulatory RNAs. These include monolayers, micelles, bilayers and vesicles. Vesicles, such as liposomes, have attracted great interest because of their specificity and the duration of action they offer from the standpoint of drug delivery. As used herein, the term “liposome” means a vesicle composed of amphiphilic lipids arranged in a spherical bilayer or bilayers.

Liposomes can be unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, can be taken up by macrophages and other cells in vivo.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G.sub.M1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known. Papahadjopoulos et al. (Ann. N.Y. Acad. Sci., 1987, 507, 64) reported the ability of monosialoganglioside G.sub.M1, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G.sub.M1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Many liposomes comprising lipids derivatized with one or more hydrophilic polymers, and methods of preparation thereof, are known. Sunamoto et al. (Bull. Chem. Soc. Jpn., 1980, 53, 2778) described liposomes comprising a nonionic detergent, 2C1215G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A number of liposomes comprising nucleic acids are known. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

A regulatory RNA described herein can be fully encapsulated in a lipid formulation, e.g., to form a SPLP, pSPLP, SNALP, or other nucleic acid-lipid particle. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs can contain a cationic lipid, a non-cationic lipid, and a lipid that prevents aggregation of the particle (e.g., a PEG-lipid conjugate). SNALPs and SPLPs can be extremely useful for systemic applications, as they exhibit extended circulation lifetimes following intravenous (i.v.) injection and accumulate at distal sites (e.g., sites physically separated from the administration site). SPLPs include “pSPLP,” which include an encapsulated condensing agent-nucleic acid complex as set forth in PCT Publication No. WO 00/03683. The particles described herein typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964, each of which is incorporated by reference in its entirety for all purposes.

The cationic lipid may be, for example, N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydr-o-3aH-cyclopenta[d][1,3]dioxol-5-amine (ALN 100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)ami-no)ethyl)piperazin-1-yl)ethylazanediyl)didodecan-2-ol (Tech G1), or a mixture thereof. The cationic lipid may comprise from about 20 mol % to about 50 mol % or about 40 mol % of the total lipid present in the particle.

The compound 2,2-Dilinoleyl-4-dimethylaminoethyl-[1,3]-dioxolane can be used to prepare lipid-siRNA nanoparticles. Synthesis of 2,2-Dilinoleyl-4-dimethylaminoethyl-[1,3]-dioxolane is described in U.S. provisional patent application No. 61/107,998 filed on Oct. 23, 2008, which is herein incorporated by reference.

The non-cationic lipid may be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid may be from about 5 mol % to about 90 mol %, about 10 mol %, or about 58 mol % if cholesterol is included, of the total lipid present in the particle.

The conjugated lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles may be from 0 mol % to about 20 mol % or about 2 mol % of the total lipid present in the particle.

The lipid to drug ratio (mass/mass ratio) (e.g., lipid to dsRNA ratio) can be in the range of from about 1:1 to about 50:1, from about 1:1 to about 25:1, from about 3:1 to about 15:1, from about 4:1 to about 10:1, from about 5:1 to about 9:1, or about 6:1 to about 9:1.

### Methods for Inhibiting Expression of BANCR

In an aspect, the description provides a method for inhibiting the expression of BANCR in a mammal. The method includes administering a composition disclosed above to the mammal such that expression of the target BANCR gene in a target tissue and/or cell is silenced.

When the organism to be treated is a mammal such as a human, the composition may be administered by any means known in the art including, but not limited to oral or parenteral routes, including intracranial (e.g., intraventricular, intraparenchymal and intrathecal), intravenous, intramuscular, subcutaneous, transdermal, airway (aerosol), nasal, rectal, and topical (including buccal and sublingual) administration. The compositions can be administered by intravenous infusion or injection.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1: Inhibition of BANCR by siRNA

The hESC-cardiomyocyte migration models described in Wilson et al., Develop. Cell 54:1-16 (2020), which is incorporated by reference in its entirety for all purposes, is used in this Example. BANCR is overexpressed in cardiomyocytes using a lentiviral vector to introduce a construct into the cardiomyocytes that overexpresses BANCR. Circular micropattern arrays can model embryonic germ layer patterning and cardiogenesis. hESC-cardiomyocytes with and without siRNA for BANCR are grown in the arrays. The siRNA are double-stranded RNAs that target SEQ ID NOs: 1, 2, or 5.

hESC-cardiomyocytes with the siRNA for BANCR show reduced migration potential compared to the control with no siRNA.

### Example 2: Targeting BANCR with siRNA

siRNAs (double-stranded RNA) with differing sequence homology to BANCR RNA (e.g., SEQ ID NO: 8-10) are transfected into human induced pluripotent stem cell-derived cardiomyocytes. After transfection, the degree of BANCR inhibition in each experiment is determined by quantitative PCR using BANCR-specific primers.

The BANCR siRNAs target one of SEQ ID NOs: 8-10 below:

(SEQ ID NO: 8)
CTCTATTGGA ATCAGCTAGC AACCACA
(exon 3)
(SEQ ID NO: 9)
CTGCTGAGAA GTTCAGAGTC AAACCTG
(exon 4)
(SEQ ID NO: 10)
GAATAATGTA AAACTCTGGA TCAATAT
(exon 3)

For example, an siRNA targeting SEQ ID NO: 8 can have the sequence:

(SEQ ID NO: 11)
GAGAUAACCU UAGUCGAUCG UUGGUGU (exon 3)

An siRNA targeting SEQ ID NO: 9 can have the sequence:

(SEQ ID NO: 12)
GACGACUCUU CAAGUCUCAG UUUGGAC (exon 4)

And an siRNA targeting SEQ ID NO: 10 can have the sequence:

(SEQ ID NO: 13)
CUUAUUACAU UUUGAGACCU AGUUAUA (exon 3)

The siRNAs can have either T or U at the U positions. The siRNAs can be made double-stranded prior to introduction to the cells by associating SEQ ID NOs: 11, 12 or 13 with the complementary RNA sequence of SEQ ID NOs: 14, 15 or 16, respectively (pairs of SEQ ID NO: 11 and 14, 12 and 15, and 13 and 16):

(SEQ ID NO: 14)
CUCUAUUGGA AUCAGCUAGC AACCACA
(exon 3)
(SEQ ID NO: 15)
CUGCUGAGAA GUUCAGAGUC AAACCUG
(exon 4)
(SEQ ID NO: 16)
GAAUAAUGUA AAACUCUGGA UCAAUAU
(exon 3)

siRNAs reduce migration of the cardiomyocytes in this model of cardiomyocyte development. See Protze et al, Human pluripotent tem cell-derived cardiovascular cells: from developmental biology to therapeutic applications, Cell Stem Cell 25:311-327 (2019), which is incorporated by reference in its entirety for all purposes.

Those skilled in the art will recognize, or be able to ascertain using no more than routine experimentation, many equivalents to the specific embodiments of the invention described herein. Such equivalents are intended to be encompassed by the following claims.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

1. An siRNA that binds to SEQ ID NO: 1 and reduces an amount of a BANCR RNA in a cell

2. The siRNA of claim 1, wherein the siRNA binds to SEQ ID NO: 2.

3. The siRNA of claim 1, wherein the siRNA has at least 15 contiguous nucleotides from SEQ ID NO: 3, or SEQ ID NO: 4.

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. A method, comprising the steps of: providing a cardiomyocyte with a siRNA that binds to SEQ ID NO: 1; and reducing the migration of the cardiomyocyte.

16. The method of claim 15, wherein the siRNA binds to SEQ ID NO: 2.

17. The method of claim 16, wherein the siRNA has at least 15 contiguous nucleotides of SEQ ID NO: 3 or SEQ ID NO: 4.

18. The method of claim 16, wherein the siRNA binds to SEQ ID NO: 8 or SEQ ID NO: 10.

19. The method of claim 16, wherein the siRNA binds to SEQ ID NO: 5.

20. The method of claim 19, wherein the siRNA has at least 15 contiguous nucleotides of SEQ ID NO: 6 or SEQ ID NO: 7.

21. The method of claim 19, wherein the siRNA binds to SEQ ID NO: 9.

22. The method of claim 16, wherein the siRNA is SEQ ID NO: 11.

23. The method of claim 22, wherein the siRNA is associated with SEQ ID NO: 14.

24. The method of claim 16, wherein the siRNA is SEQ ID NO: 13.

25. The method of claim 24, wherein the siRNA is associated with SEQ ID NO: 16.

26. The method of claim 19, wherein the siRNA is SEQ ID NO: 14.

27. The method of claim 26, wherein the siRNA is associated with its complement of SEQ ID NO: 15.

28. The method of claim 15, wherein the siRNA reduces migration of a hESC-cardiomyocyte.

29. The method of claim 22, wherein the siRNA reduces migration of a hESC-cardiomyocyte.

30. The method of claim 24, wherein the siRNA reduces migration of a hESC-cardiomyocyte.

31. The method of claim 26, wherein the siRNA reduces migration of a hESC-cardiomyocyte.
